(19) United States
(12) Patent Application Publication
(10) Pub. No.: US 2008/0200565 A1
(43) Pub. Date: Aug. 21, 2008

(54) Surfactant-only microemulsions for cleaning system design and product delivery

(76) Inventors: Jeffrey H. Harwell, Norman, OK (US); David A. Sabatini, Norman, OK (US); William Shaw, Irving, TX (US); Mrinal Gala, Houston, TX (US); Bor-Jier Shiau, Norman, OK (US); Tzu-Ping Hsu, Norman, OK (US)

Correspondence Address: DUNLAP CODDING & ROGERS, P.C., PO BOX 16370, OKLAHOMA CITY, OK, 73113, US

(21) Appl. No.: 12/011,567

(22) Filed: Jan. 28, 2008

Related U.S. Application Data
(60) Provisional application No. 60/897,709, filed on Jan. 26, 2007.

(51) Int. Cl. A61K 47/14; C11D 3/20; B08B 3/04; C09K 8/60

(52) U.S. Cl. 514/785; 510/537; 134/10; 134/26; 8/137; 507/260

(57) ABSTRACT

Surfactant systems are provided that, upon contact with an oil, can produce a Windsor Type III middle phase microemulsion at a total surfactant concentration of 1.5% to 1.0% or less based on the weight of water in the surfactant system, without the need for a cosolvent or linking molecule. The microemulsions can have a separation time less than about 2 hours and even less than about 15 minutes.

### CROSS REFERENCE TO RELATED APPLICATIONS

This application claims the benefit under 35 U.S.C. 119(e) of U.S. Provisional Application Ser. No. 60/897,709, filed Jan. 26, 2007, the contents of which are hereby expressly incorporated by reference herein in its entirety.

### STATEMENT REGARDING FEDERALLY SPONSORED RESEARCH OR DEVELOPMENT

Not applicable.

### BACKGROUND OF THE INVENTION

1. Field of the Invention

The present invention relates generally to effective remediation, cleaning and product delivery systems and, more particularly, but not by way of limitation, to surfactant mixtures capable of producing microemulsions without the need for a cosolvent or linking molecule.

2. Brief Description of the Prior Art

Microemulsions are thermodynamically stable oil/water dispersions that can be used as cleaning systems and product delivery systems. Of particular interest are Winsor Type III or “middle phase” microemulsions, where the oil/water interfacial tension between oil and water is ultra-low. Example applications of microemulsions include detergent formulations, industrial cleaning systems, enhanced oil recovery, surfactant enhanced aquifer remediation, and drug delivery systems.

Traditional microemulsion formulations require on the order of 1 to 10 weight percent surfactant, together with a cosolvent such as an alcohol or hydrotrope to obtain desirable phase behavior, prevent formation of undesirable phases, and obtain ultralow interfacial tensions and high coalescence rates. The concentration of the cosolvent generally matches or greatly exceeds the concentration of the surfactant.

It is generally accepted in the art that surfactant-only microemulsions are not possible or are not useful due to high concentration requirements, long equilibration times, high cost, and/or poor biodegrability. The state of the art in microemulsion design today still assumes use of a cosolvent such as low molecular weight alcohol or hydrotrope, or a linking molecule such as a hydrophilic linker or lipophilic linker, to achieve the desired phase behavior. Previous attempts to formulate useful surfactant-only microemulsions, as summarized briefly below, have not been commercially successful.

For example, Baran et al., (Baran et al., 1994a, 1994b) developed microemulsion formulations for different chlorinated solvents using the single surfactant system sodium dihexyl sulfossucinate (AEROSOL MA, referred to herein as MA) and/or binary surfactant systems with the MA surfactant combined with other surfactants selected from a variety of Guerbet ethoxy (EO) and propoxy (PO) sulfates. Examples of these surfactants included C₁₄GA(EO)ₓSO₄Na (x=1.6 and 2.9), C₁₄EX(EO)₄SO₄Na, C₁₆EX(EO)_{y}SO₄Na (y=0, 2, 4, 6, 8, and 10), and C₁₆EX(PO)_{z}SO₄Na (z=2.6, 4, and 6.5). Baran et al., (1994a) were able to achieve the targeted microemulsion with low interfacial tension (IFT), or so called Winsor Type III (or middle-phase microemulsion) or low IFT microemulsion, for the chlorinated solvents, such as tetrachloroethylene (PCE) and trichloroethylene (TCE). However, the surfactant concentrations used were either very high (much greater than 1 wt %) or the co-surfactants used, including the sulfated Guerbet alcohol ethoxylate and propoxylate surfactants, were mostly experimental chemicals, specially developed for their laboratory (in some cases they were high-performance surfactants) having higher chemical costs compared to other surfactants readily available commercially.

Pennell et al., (1994) investigated use of binary surfactant systems for creating low IFT microemulsion for tetrachloroethylene (PCE). A 4 wt % surfactant mixture of sodium diamyl sulfosuccinate (MY) and sodium dioctyl sulfosuccinate (AOT) was used to produce low IFT microemulsion for PCE contaminant. Lower surfactant concentrations were not reported. Pennell et al. also used a binary surfactant mixture of sodium dihexyl sulfosuccinate and sodium dioctyl sulfosuccinate (at 4% solution); however, the resulting microemulsion was a much less effective Winsor Type I (oil-in-water microemulsion) with a higher IFT. Such microemulsions are much less effective for contaminant recovery while, at the same time, minimizing the potential risk of uncontrolled vertical migration of PCE in the subsurface.

Wellington and Richardson (1995) evaluated a series of surfactant mixtures using NEODOL Propoxy Ethoxy Glyceryl Sulfonates as the main surfactant to achieve an adequately low IFT to improve oil recovery without the formation of microemulsions. In their study, Wellington and Richardson pointed out that “In the context of the method described in this paper, microemulsion formation was usually undesirable since surfactant lag increased and oil or soil contaminant recovery tailed over and extended production volume.” Therefore, the Wellington and Richardson study provides an alternative approach to accomplishing high oil recovery, specifically reducing the IFT to lower level but without producing a microemulsion or the very low IFTs associated with microemulsions. The results of their study also indicate that a surfactant mixture combining anionic and cationic surfactants does not readily generate low IFT microemulsions.

Jayanti et al. (2001) also studied a binary system of anionic and cationic surfactant mixtures. Examples of the anionic surfactants used by Jayanti et al., were sodium C₁₂-C₁₃ (propylene oxide)₄ sulfate and the sodium C₁₂ (PO)₃ sulfate. The cationic surfactant was a propoxylated quarternary ammonium chloride, commercially sold as Variquat CC-9. A mixture of 0.4 wt % C₁₂(PO)₃ and 0.2 wt % Variquat CC-9 was used to obtain a low IFT microemulsion in one of their tests (FIGS. 2 and 3 in the article). However, only a much more concentrated surfactant mixture (4% C₁₂-C₁₃(PO)₄SO₄Na and 0.5% Variquat CC-9 plus 8% isobutanol) was further tested in the one-dimensional soil column study. With addition of the cationic surfactant (Variquat CC-9), Jayanti et al. was able to achieve equilibration times of 15 to 20 hours, even in the absence of an alcohol cosolvent. However, these equilibration times are still much longer than desirable. Also, use of cationic surfactants is not desirable for many applications because they tend to be less biodegradable (commonly used as a biocide in household cleaning formulations), and they have much higher sorption losses in silicate sands compared to anionic and nonionic surfactants.

Sabatini et al. (2000) conducted a series of surfactant screening batch and column tests for remediation of PCE. Of the surfactants screened, a mixture of branched alkyl (C₁₄-C₁₅) propyloxylated sulfate (Isalchem 145-4PO-SO₄) and mono- and di-hexadecyl di-phenyloxide di-sulfonate (Dowfax 8390) (3% total surfactant concentration) was the best for achieving a low IFT microemulsion and high oil (PCE) recovery in the 1-D column. However, the 3% total surfactant concentration was higher than desirable, especially because the branched alkyl (C₁₄-C₁₅) propyloxylated sulfate was an experimental surfactant and therefore more costly and not readily available in large quantities for field implementation.

Sabatini et al. (2000) also evaluated a mixture of 2.5% Dowfax/2.5% sodium dioctyl sulfosuccinate (AOT)/2.5% tartaric acid (a lipophilic linker) for PCE removal. Interestingly, they found out that this surfactant system could achieve low IFT microemulsion in the batch experiment, but further testing in the 1-D column was less desirable due to column plugging as a result of colloidal dispersion by the surfactant.

Others have explored mixtures of anionic and nonionic surfactants to achieve the low IFT microemulsion. For example, Wu et al. (2000) used a mixture of sodium dioctyl sulfosuccinate (AOT) (1.2 wt %) and nonionic surfactants, POE (20) sorbitan monostearate-TWEEN® 60 (1.1 wt %) or POE (20) sorbitan monooleate-TWEEN® 80 (1.1 wt %), to produce a low IFT microemulsion. However, mixed anionic and nonionic surfactant systems tend to have higher surfactant losses due to surfactant adsorption, especially of the nonionic surfactant, onto the soil matrix. In remediation efforts for example, higher surfactant losses require multiple pore volumes of surfactant injection and increase the remediation costs necessary to accomplish the same remediation goal, as indicated in a pilot-scale surfactant flush (Sabatini et al., 1998, 2005). Also, the viscosities of mixed anionic and nonionic surfactant solution are typically greater than binary mixtures of anionic surfactants under similar conditions. During site remediation, excessively high surfactant solution viscosity can reduce the injection rates and increase the injection pressure, which will typically increase the remediation costs. Also, higher injection pressure makes remediating shallow contaminations more difficult due to less pressure head for delivering the surfactant solution (Sabatini et al., 2005).

A final example of a microemulsion based on a mixture of two surfactants, without use of a cosolvent or linking molecule, is found in U.S. Pat. Nos. 6,913,419 and 7,021,863 entitled “IN-SITU SURFACTANT AND CHEMICAL OXIDANT FLUSHING FOR COMPLETE REMEDIATION OF CONTAMINANTS AND METHODS OF USING SAME” (referred to herein as the Shiau patents), both of which are incorporated herein by reference. The Shiau patents disclose improved binary surfactant systems, including sodium dioctyl sulfosuccinate (AOT) and mono- and di-hexadecyl di-phenyloxide di-sulfonate (Dowfax 8390), to create low IFT microemulsion without adding the hydrotrope (such as Shiau et al., 1995) or lipophilic linker, such as tartaric acid (Sabatini et al., 2000). However, one of the components of the improved binary surfactant systems disclosed in the Shiau patents is less biodegradable than desirable for environmental applications; therefore, improved surfactant-only microemulsion formulations are still needed.

In summary, while surfactant-only microemulsion formulations have been found, such mixtures have typically required high surfactant concentrations and long equilibrium times. In addition, these mixtures often have poor biodegrability and high surfactant losses due to surfactant adsorption. Thus, there is a continuing need for surfactant mixtures and microemulsions having improved performance properties and cost benefits, as well as methods for using the improved surfactant mixtures and microemulsions formed therefrom.

### SUMMARY OF THE INVENTION

The invention provides a surfactant system including sodium bis (2-ethylhexyl)sulfosuccinate surfactant, a laureth sulfate anionic surfactant, and water. The sodium bis(2-ethylhexyl)sulfosuccinate and the laureth sulfate surfactants are present in the system in concentrations effective to produce a microemulsion, without the need for a cosolvent or linking molecule, when the surfactant system is combined with an oil. Further, the sodium bis(2-ethylhexyl)sulfosuccinate plus laureth sulfate surfactant system can produce a Windsor Type III middle phase microemulsion, without the need for a cosolvent or linking molecule, at a total surfactant concentration of 1% or less based on the weight of water in the surfactant system.

Sodium dihexyl sulfosuccinate can be added to the sodium bis (2-ethylhexyl)sulfosuccinate plus laureth sulfate surfactant system to produce a surfactant system that, upon contact with an oil, can produce a Windsor Type III middle phase microemulsion at a total surfactant concentration of 1.5% to 1.0% or less based on the weight of water in the surfactant system, without the need for a cosolvent or linking molecule, and the microemlsion can have a separation time less than about 2 hours and even less than about 15 minutes.

Similarly, sodium diamyl sulfosuccinate can be added to the sodium bis(2-ethylhexyl)sulfosuccinate plus laureth sulfate surfactant system to produce a surfactant system that, upon contact with an oil, can produce a Windsor Type III middle phase microemulsion at a total surfactant concentration of 1.5% to 1.0% or less based on the weight of water in the surfactant system, without the need for a cosolvent or linking molecule, and the microemulsion can have a separation time less than about 2 hours and even less than about 15 minutes.

The invention further provides a surfactant system including water and a surfactant mixture, wherein the surfactant mixture can be sodium bis(2-ethylhexyl)sulfosuccinate, sodium dihexyl sulfosuccinate and a third surfactant. The third surfactant can be a branched alkyl (C₁₄-C₁₅) propyloxylated sulfate surfactant, sodium diamyl sulfosuccinate, polyoxyethylene (20) sorbitan monooleate, polyoxyethylene (20) sorbitan monostearate, or mixtures thereof. Again, the surfactants are present in the surfactant system in concentrations effective to produce a microemulsion, without the need for a cosolvent or linking molecule, when the surfactant system is combined with an oil. In addition, the surfactant systems can produce the microemulsions without the need for a cosolvent or linking molecule, when the surfactant system is combined with an oil at total surfactant concentrations of 1.5% to 1.0% or less based on the weight of water in the surfactant system, and the microemulsion can have a separation time less than about 2 hours and even less than about 15 minutes.

Methods are provided for substantially removing organic contaminants from a subsurface formation by introducing any of the above-described surfactant systems into the subsurface formation, contacting the surfactant system with the organic contaminants to form a microemulsion, and recovering the resulting microemulsion.

A process for recovering crude oil from a subterranean oil-bearing formation is provided. The process includes injecting any of the above-described surfactant systems into the oil-bearing formation, wherein the surfactants combine with the crude oil to form a microemulsion without the aid of a cosolvent or linking molecule. A drive fluid is injected into the formation to drive the crude oil toward one or more recovery wells and the crude oil is recovered via the recovery wells.

A process for cleaning organic contaminants from a hard surface is also provided and includes contacting the hard surface with an effective amount of any of the above-described surfactant systems.

Additionally, a method for laundering fabrics having organic contaminants is provided. The method includes the step of diluting an aqueous liquid composition in its neat form into an aqueous bath. The aqueous liquid composition contains a concentrated surfactant mixture of the type described above. Upon dilution in the aqueous bath, the surfactants become present in the aqueous bath in concentrations effective to produce a microemulsion, without the need for a cosolvent or linking molecule, when the surfactant system is contacted with the organic contaminants.

Still further, a method for intravenous drug delivery is provided. In this method, microdroplets of the drug are prepared with any of the surfactant systems described above.

Thus, utilizing (1) the technology known in the art; (2) the above-referenced general description of the presently disclosed and claimed inventive process(es), methodology(ies), apparatus(es) and composition(s); and (3) the detailed description of the invention that follows, the advantages and novelties of the presently disclosed and claimed inventive process(es), methodology(ies), apparatus(es) and composition(s) would be readily apparent to one of ordinary skill in the art.

### BRIEF DESCRIPTION OF THE DRAWINGS

FIG. 1 is a pictorial representation showing, generally, the formation of microemulsions of the present invention.

FIG. 2 is a schematic and pictorial representation showing phase behavior of the microemulsions of FIG. 1.

FIG. 3 is a schematic representation comparing the selection of surfactants in the present invention to the surfactant selection in U.S. Pat. Nos. 6,913,419 and 7,021,863.

FIG. 4 is a table showing characteristics of the surfactants of FIG. 3.

FIG. 5 is a schematic representation showing the methodology for phase behavior.

FIG. 6 is a graphical representation showing a phase diagram utilizing Decane-SEHSS-DPDS, the surfactant system in U.S. Pat. Nos. 6,913,419 and 7,021,863.

FIG. 7 is a graphical representation showing a fish diagram of Decane-SEHSS-SDES-1.

FIG. 8 is a graphical representation showing a fish diagram of Decane-SEHSS-SDES-2.

FIG. 9 is a graphical representation showing a fish diagram of Decane-SEHSS-SDES-3.

FIG. 10 is a graphical representation showing a fish diagram of Decane-SEHSS-SDES-3.5.

FIG. 11 is a table showing the results based on fish diagrams of FIGS. 7-10.

### DETAILED DESCRIPTION OF THE INVENTION

Before explaining at least one embodiment of the invention in detail, it is to be understood that the invention is not limited in its application to the details of construction, experiments, exemplary data, and/or the arrangement of the components set forth in the following description or illustrated in the drawings. The invention is capable of other embodiments or of being practiced or carried out in various ways. Also, it is to be understood that the phraseology and terminology employed herein is for purpose of description and should not be regarded as limiting.

Conventionally, microemulsion formulations require on the order of 1 to 10 weight percent surfactant, together with a cosolvent such as an alcohol, or linking molecules such as hydrotopes, in order to obtain desirable phase behavior, prevent formation of undesirable phases, and obtain ultralow interfacial tensions and high coalescence rates. Example alcohols used conventionally are isobutanol and isopropyl alcohol, although many other alcohols have and could be used for microemulsion formation. Linker molecules are additives used to enhance the surfactant-oil interaction (lipophilic linkers) or the surfactant-water interaction (hydrophilic linkers) and thus enable microemulsion formation. For example, alkyl naphthalene sulfonates are hydrotropic linker molecules frequently used to enable microemulsion formation with anionic surfactants.

The concentration of the cosolvent or linking molecules generally matches or greatly exceeds the concentration of the surfactant. The surfactant mixtures capable of producing a Winsor Type III or Winsor Type IV oil/water microemulsion typically require from 5 to 15 wt % cosolvent or linking molecule.

The surfactant-only microemulsion system, disclosed in the Surbec patents, produces advantageous phase behavior over a wide range of oils and conditions, working at both room temperature and at temperatures in the subsurface. One of the components of the formulation disclosed in the Surbec patents—a commercial surfactant blend with the trade name of CalFax or DowFax—is a unique molecule that has no close analogues in any other commercial surfactant; yet this surfactant is not as readily biodegradable as seems desirable for application in environmental remediation. Initially, no substitute for the less biodegradable component of the surfactant system could be found.

The present invention describes new surfactant mixtures having improved biodegrability and capable of producing microemulsions containing less than 1.5 or less than 1.0 weight percent surfactant without the need for a cosolvent or linking molecule. In addition, the binary and ternary surfactant formulations presented provide performance improvement as well as cost benefits resulting from low surfactant dosage compared to prior art formulations.

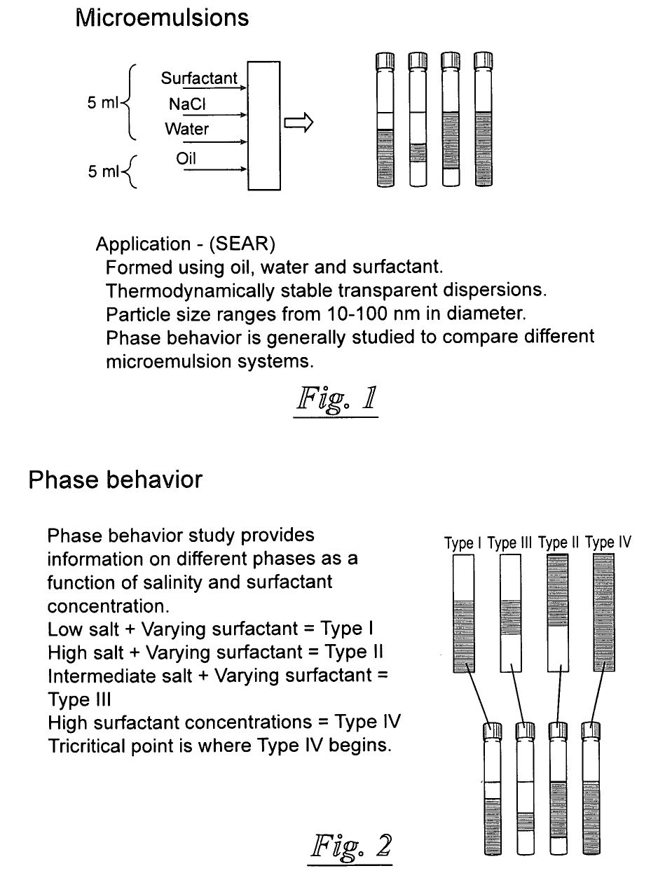

Referring now to the drawings, and more particularly to FIG. 1, shown therein is a formation of surfactant-only microemulsions from binary or ternary surfactant mixtures applied for use in surfactant-enhanced aquifer remediation. The microemulsions are formed using oil, water and surfactant. The mixtures are thermodynamically stable oil/water dispersions. Phase behavior of the microemulsions can be studied to compare the different microemulsion systems and to establish that the dispersions are at thermodynamic equilibrium.

As shown in FIG. 2, the phase behavior study provides information on different phases as a function of salinity and surfactant concentration. Winsor Type I is a low salt+varying surfactant. Winsor Type II is a high salt+varying surfactant. Winsor Type III is an intermediate salt+varying surfactant. Winsor Type IV is a high surfactant concentration. The tricritical point is where Winsor Type IV begins.

The surfactant systems of the present invention include at least two surfactants and can be, for example, a binary system of two surfactants or a ternary system of three surfactants. In one embodiment, a binary surfactant system includes the surfactant sodium bis(2-ethylhexyl) sulfosuccinate and a laureth sulfate anionic surfactant. Sodium bis(2-ethylhexyl)sulfosuccinate (sometimes referred to herein as SEHSS) is available commercially as Aerosol-OT (commonly referred to as AOT) from Fisher Scientific. The laureth sulfate anionic surfactant can be a sodium laureth sulfate (sodium dodecyl oxyethoxy ethyl sulfate). Suitable sodium laureth sulfate surfactants include, but are not limited to, sodium dodecyl ethoxy (1) sulfate (sometimes referred to herein as SDES-1), sodium dodecyl ethoxy (2) sulfate (sometimes referred to herein as SDES-2), sodium dodecyl ethoxy (3) sulfate (sometimes referred to herein as SDES-3), and a mixture of sodium dodecyl ethoxy (3) sulfate and sodium dodecyl ethoxy (4) sulfate (the mixture sometimes referred to herein as SDES-3.5). Sodium laureth sulfate surfactants are available commercially from, for example, Stepan Chemical Co. in Northfield, Ill.

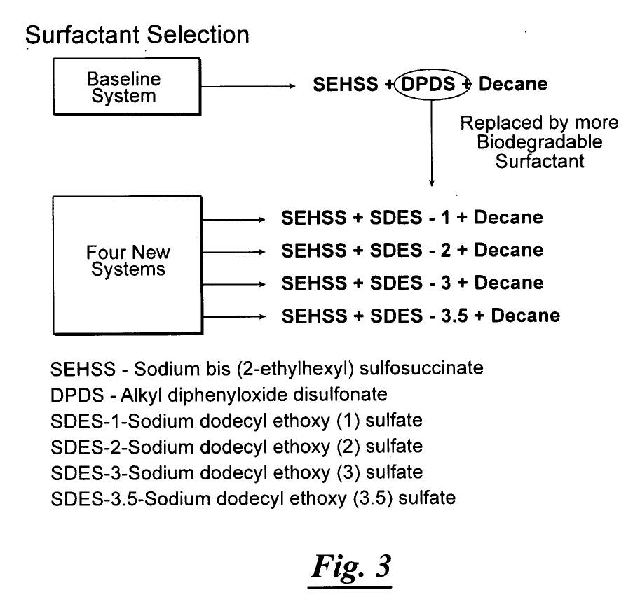

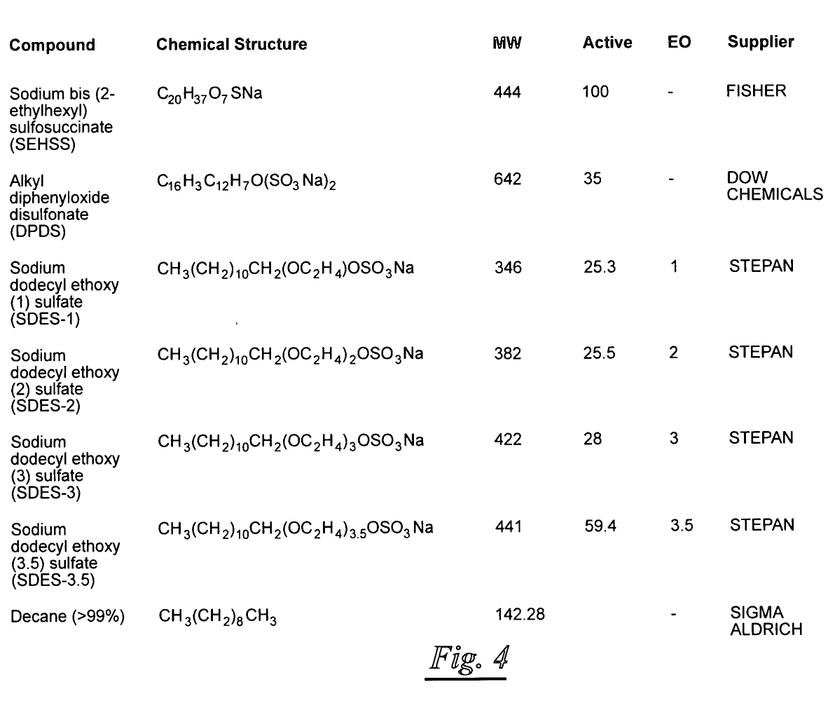

Referring now to FIG. 3, a baseline system was selected composed of SEHSS, alkyl diphenyloxide disulfonate (DPDS) and decane. This is the surfactant system disclosed in U.S. Pat. Nos. 6,913,419 and 7,021,863 and was used to compare with new systems containing sodium laureth sulfate. Sodium laureth sulfate surfactants SDES-1, SDES-2, SDES-3 and SDES-3.5 were found to perform synergistically with SEHSS. In addition, the SEHSS/SDES systems are more biodegradable than the baseline SEHSS/DPDS system. FIG. 4 shows the chemical formula, molecular weight, activity, number of ethoxy groups, and supplier for each of the surfactants and the decane utilized in the microemulsion systems shown in FIG. 3.

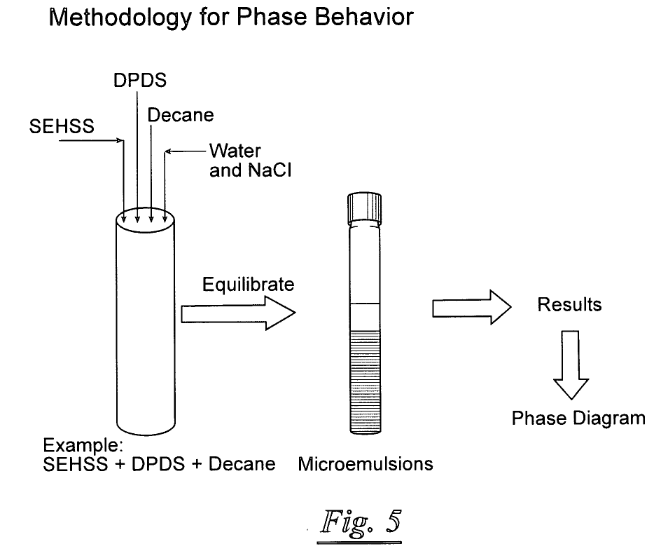

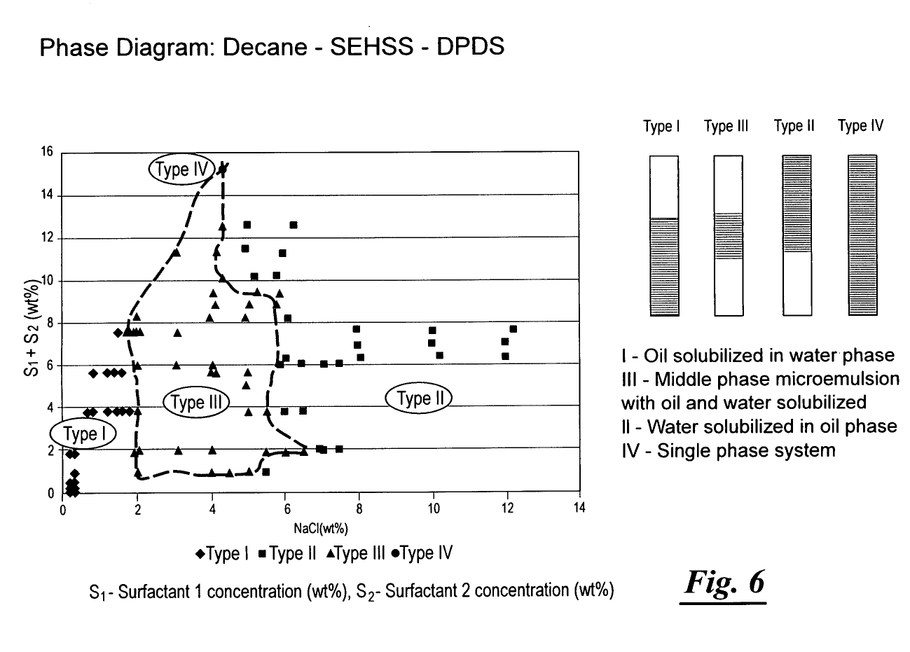

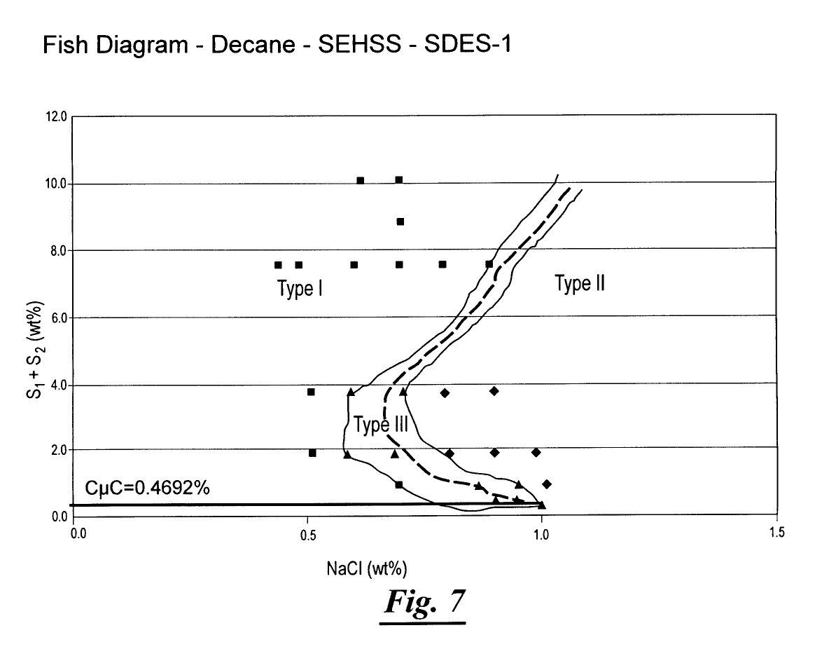

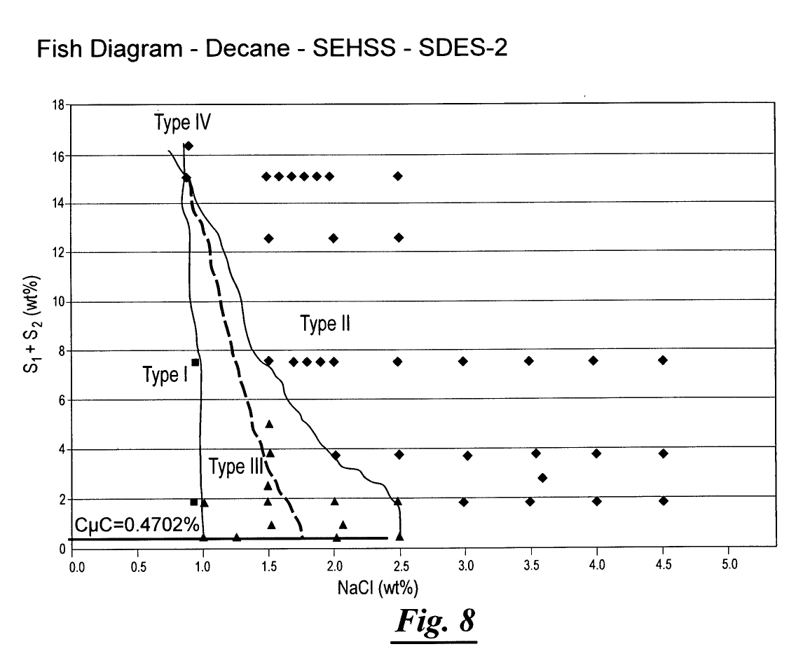

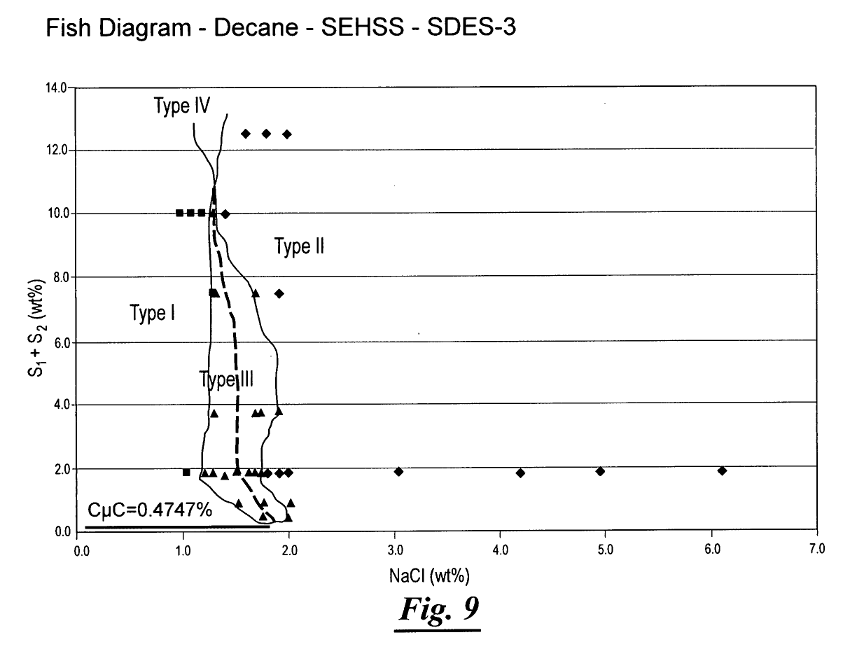

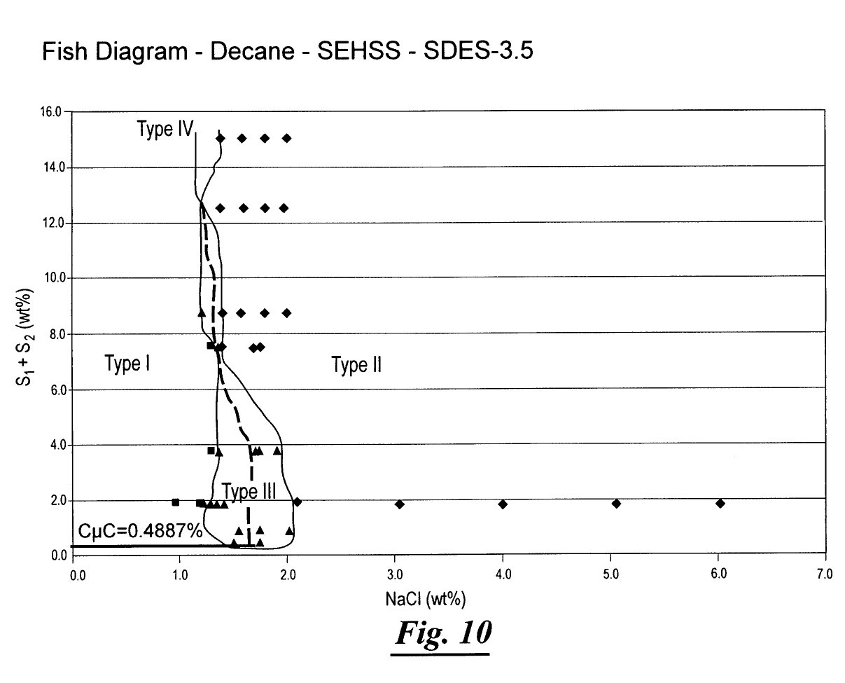

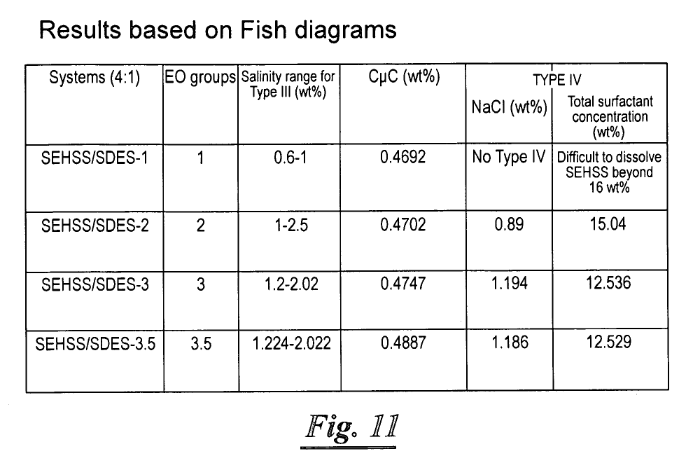

Referring to FIG. 5, the methodology for testing the phase behavior is shown. For example, the baseline system compounds of SEHSS, DPDS, decane, water and NaCl were placed in a vial and equilibrated to form a microemulsion. The results of the microemulsion were determined and a phase diagram as shown in FIG. 6 was constructed. The same methodology was performed on each of the four new systems to form fish diagrams shown in FIGS. 7-10. The Winsor Type III middle phase microemulsion appears at a specific concentration that is higher than the critical micelle concentration (CMC). This point is denoted as the critical middle phase microemulsion concentration (CμC) in FIGS. 7-10.

The results based on the fish diagrams for each of the four new systems are summarized in FIG. 11. These laboratory tests demonstrated that the aqueous SEHSS/SDES systems in FIG. 3 produce Winsor Type III microemulsions when mixed with decane and using total surfactant concentrations at or below 1.5% and even below 1.0% by weight of water in the surfactant system. Surprisingly, these microemulsions were obtained without the use of any additional reagents such as a cosolvent or linking molecule.

While decane was used as the oil in these laboratory experiments, the oil could be any hydrocarbon capable of forming an emulsion with an aqueous surfactant system. Example oils that can be used include, but are not limited to, crude oil, petroleum products, organic solvents, organic contaminants, pharmaceutical compositions, and edible oils (e.g., vegetable oil), and fats and greases. Use of surfactant microemulsions to recover crude oil and microemulsions of organic solvents are well known in the industry. Examples of petroleum products that can be solubilized as a microemulsion include, but are not limited to gasoline fuels, diesel fuels, heating oil, bunker oil, coal tars, and creosote. Heavy metal and organic contaminants that can be solubilized as a microemulsion include, but are not limited to, chromium and DNAPL contaminants in groundwater, and food deposits on hard surfaces and clothing. Recently pharmaceutical research has focused on solubilizing a variety of pharmaceutical compositions using microemulsions as a vehicle for intravenous drug delivery.

The surfactant system described in the Surbec patents (U.S. Pat. Nos. 6,913,419 and 7,021,863) has been used successfully in the field some two dozen times. The improved surfactant system described above is being tested at the prototype scale for effectiveness at producing high contaminant removal rates from soil columns.

In an effort to reduce the equilibrium or separation time for the microemulsions formed, ternary surfactant systems were tested. It was found that sodium dihexyl sulfosuccinate surfactant (sometimes referred to herein as MA), when added to the SEHSS/SDES systems, continued to behave synergistically to produce microemulsions with oil, including Winsor Type III middle phase microemulsions, using total surfactant concentrations at or below 1.5% and even below 1.0% by weight of water in the surfactant system without the need for a cosolvent or linking molecule. In addition, however, the separation time for the microemulsions formed was less than about 2 hours and can be less than about 15 minutes. The same synergistic results were obtained by addition of sodium diamyl sulfosuccinate surfactant to the SEHSS/SDES systems.

Synergistic surfactant systems were further found to include an aqueous mixture of sodium bis(2-ethylhexyl)sulfosuccinate (SEHSS), sodium dihexyl sulfosuccinate (MA), and a third surfactant, wherein the third surfactant can be a branched alkyl (C₁₄-C₁₅) propyloxylated sulfate surfactant, sodium diamyl sulfosuccinate, polyoxyethylene (20) sorbitan monooleate, polyoxyethylene (20) sorbitan monostearate, or mixtures thereof. Again, the surfactants are present in the surfactant system in concentrations effective to produce a microemulsion, without the need for a cosolvent or linking molecule, when the surfactant system is combined with an oil. In addition, the SEHSS/MA/third surfactant systems can produce the microemulsions, including Winsor Type III middle phase microemulsions, without the need for a cosolvent or linking molecule, when the surfactant system is combined with an oil at total surfactant concentrations of 1.5% to 1.0% or less based on the weight of water in the surfactant system. The microemulsions produced by these SEHSS/MA/third surfactant systems, when contacted with an oil, can have separation times less than about 2 hours and even less than about 15 minutes.

The water component of the surfactant systems described above can be fresh water or salt water. Non-limiting examples of salt water include electrolyte, brine, sea water and formation water. The water can include NaCl and CaCl₂. Typically NaCl is present in amounts up to about 5 wt % based on the total weight of water in the surfactant system, and CaCl₂ is present in amounts up to about 1 wt % based on the total weight of water in the surfactant system. The water can include other salts such as MgCl₂, carbonates, and other chemicals either naturally present or as system additions.

It should be understood that the present invention may be used for various applications. For example, the present invention may be used in environmental remediation. Surfactant mixtures may be designed to release contaminants trapped by capillary forces in a subsurface environment through a reduction of the interfacial tension between the aquifer water and the trapped contaminant liquid. In environmental remediation, the present microemulsion allows a reduction in the concentration of chemicals required. The reduction produces a substantial cost savings and produces a competitive advantage relative to other remediation technologies. Thus, methods are provided for substantially removing organic contaminants from a subsurface formation by introducing any of the above-described surfactant systems into the subsurface formation, contacting the surfactant system with the organic contaminants to form a microemulsion, and recovering the resulting microemulsion.

Additionally, the present invention may be used in enhanced oil recovery. Surfactant mixtures may be designed to release oil trapped by capillary forces in an oil reservoir through reduction in the interfacial tension between the reservoir water and trapped oil. In enhanced oil recovery, the novel microemulsion design methodology described herein allows a reduction in the concentration of chemicals required, and should produce a substantial reduction in the cost of this enhanced oil recovery technology, possibly a critical reduction in the cost if this technology is ever to be commercially viable.

Thus the above-described surfactant systems can be used advantageously to recover crude oil from a subterranean oil-bearing formation. Such a process includes injecting the surfactant system into the oil-bearing formation, wherein the surfactants combine with the crude oil to form a microemulsion without the aid of a cosolvent or linking molecule. A drive fluid can be injected into the formation to drive the crude oil toward one or more recovery wells, and the crude oil can be recovered via the recovery wells.

Further, the microemulsions of the present invention may be used in commercial cleaning technologies. Surfactant-based cleaning formulations show improved performance under the conditions close to those that produce the Winsor Type III and Winsor Type IV microemulsions described herein. Therefore, the same approach that was used to design a surfactant system for increasing effectiveness and reducing cost in an environmental remediation application should also produce increased effectiveness and reduced cost in industrial and household cleaning formulations such as laundry detergents and household cleaners. The result could be very important as consumer and industrial and institutional product companies reformulate their products in response to increased chemical prices resulting from the increase in oil prices. Formulation of the microemulsion without addition of low molecular weight alcohols allows stable Type III microemulsion without high concentrations of volatile organic compounds (VOCs) that present air pollution problems.

Thus, processes for cleaning organic contaminants from a hard surface can include contacting the hard surface with an effective amount of any of the above-described surfactant systems. Also, methods for laundering fabrics having organic contaminants can include the step of diluting an aqueous liquid composition in its neat form into an aqueous bath, wherein the aqueous liquid composition can contain a concentrated surfactant mixture of the types described above. Upon dilution in the aqueous bath, the surfactants become present in the aqueous bath in concentrations effective to produce a microemulsion, without the need for a cosolvent or linking molecule, when the surfactant system is contacted with the organic contaminants present on the fabrics to be laundered.

Microemulsions are also very effective as drug delivery vehicles for drug delivery systems. The elimination of alcohols and reduction of concentrations of chemicals in the design of microemulsion-based drug delivery systems should be applicable to producing such formulations with reduced toxicity and volatility. Methods for intravenous drug delivery can include preparing microdroplets of the drug with any of the surfactant systems described above.

Without limiting the scope of the present invention, it is postulated that the formulations described herein require a combination of at least one surfactant having a larger ratio of volume of a hydrophobic moiety to an interfacial area occupied by the hydrophilic moiety and the length of the hydrophobic moiety (dimensionless packing factor) together with a second surfactant having a smaller dimensionless packing factor. The dimensionless packing factor of the mixture may change because of an addition of electrolyte and/or variation of the ratio of the larger packing factor surfactant and the smaller packing factor surfactant. It is also postulated that the surfactant mixture should obtain an average packing factor of approximately 1 and that many surfactants currently used in commercial cleaning systems will not be able to produce microemulsions by the approach described herein because many have too small a packing factor.

Theoretically, if the key to obtaining surfactant-only microemulsions is to mix surfactants with different dimensionless packing factors, then the behavior of a mixture may be adjusted by adjusting the area per head group with an electrolyte or the use of a nonionic surfactant or an amphoteric or zwitterionic surfactant, or the attractive interaction between the head groups, in order to obtain an average dimensionless packing factor of approximately 1, which may then produce a lamellar surfactant membrane.

Regardless of the actual mechanism, however, it has been shown that the surfactant systems described herein can be used to form Winsor III microemulsions using concentrations of surfactant at or below 1% (based on the weight of water in the system), with ultralow oil/water interfacial tensions, without the need for adding an alcohol, hydrotrope, fatty acid, or any other cosolvent or linking molecule to modify the phase behavior.

In order to further illustrate the surfactant mixtures, systems, and methods of the present invention, the following examples are given.

### EXAMPLE 1

Early attempts were made to repeat Baran et al., tests (Baran et al., 1994a, 1994b) using lower surfactant concentrations, but without success. We found that when the MA-only surfactant system was reduced in concentration to approximate 1.5 wt % surfactant, the low IFT microemulsion systems with such contaminants as PCE, TCE or petroleum gasoline fuels quickly disappeared, thus causing the system to lose effectiveness. For example, the MA-only surfactant system at concentrations less than 1.5 wt % would not retain the ability to release oil from soil grains. One possibility for losing the formation of the low IFT microemulsion may be due to the very high critical micelle concentration of MA (concentrations ranging between 1.2 wt % to 1.5 wt % in common ambient and groundwater conditions). In any event, the microemulsions formed using the single MA surfactant at low concentrations was not stable.

### EXAMPLE 2

Though the low IFT microemulsions observed in the new binary anionic surfactants and decane were encouraging, we also found that the equilibration times for the low IFT microemulsions in these new binary surfactant mixtures (SEHSS/SDES-1, SEHSS/SDES-2, SEHSS/SDES-3 SEHSS/SDES-3.5) were less impressive than Shiau's system (U.S. Pat. Nos. 6,913,419 and 7,021,863) under ambient conditions, taking several hours to equilibrate instead of few minutes to one to two hours. Therefore, we further investigated the possibility of adding a second cosurfactant to the binary system (or so called ternary surfactant system) to improve the equilibration rate of the microemulsion. One should note that the Dowfax or Calfax surfactant used in Shiau's patents is actually a surfactant mixture including at least two different surfactants with different molecular structures. Therefore, in reality, Shiau's low IFT system is a ternary surfactant mixture. Representative results from further improvements made using the ternary surfactant mixtures are also provided herein.

A ternary surfactant mixture solution, prepared in either deionized water or actual contaminated site groundwater, included sodium dioctyl sulfosuccinate (SEHSS, or AOT), sodium dihexyl sulfosuccinate (MA), and sodium laureth sulfate SDES-3 (modified). The SDES-3 used was STEOL® KS-460 from the Stepan Company. As shown in Table 1 below, we were able to achieve the low IFT microemulsions for the weathered gasoline fuels using a salt additive, NaCl, between 1.4% to 1.9% concentrations. Note that the equilibration times for the resulting low IFT microemulsion (denoted as Type III system) are typically in minutes. Further improvement made was that all the total surfactant concentrations were between 0.56 wt % to 0.68 wt %. In this example, all three surfactants, SEHSS, MA, and STEOL® KS-460 are anionic and readily biodegraded in the environment. Based on this example, a mixture of sodium dioctyl sulfosuccinate (SEHSS), sodium dihexyl sulfosuccinate (MA), and sodium laureth sulfate SDES-3 (modified), or STEOL® KS-460 provided further improvement in the equilibration (or separation) time for the low IFT microemulsions as compared to the binary system, dioctyl sulfosuccinate and sodium laureth sulfate SDES 3 (modified), or STEOL® KS-460 or other STEOL® surfactants presented in the present invention.

TABLE 1

| | | SDES-3 | | | |
|---|---|---|---|---|---|
| SEHSS | MA | (modified), | NaCl | Winsor | |
| (wt %) | (wt %) | (wt %) | (wt %) | Type | Note |
| System I* | | | | | |
| 0.2 | 0.3 | 0.06 | 1 | I | Took about 30 |
| | | | | | min to separate |
| 0.2 | 0.3 | 0.06 | 1.4 | III | Quick |
| | | | | | separation** |
| 0.2 | 0.3 | 0.06 | 1.9 | III | Quick |
| | | | | | separation |
| 0.2 | 0.3 | 0.06 | 2.1 | II | Quick |
| | | | | | separation |
| System II* | | | | | |
| 0.25 | 0.37 | 0.06 | 1 | I | Took about 30 |
| | | | | | min to separate |
| 0.25 | 0.37 | 0.06 | 1.5 | III | Quick |
| | | | | | separation |
| 0.25 | 0.37 | 0.06 | 2 | II | Quick |
| | | | | | separation |

All surfactant systems were tested with a weathered gasoline from an actual site, L*.
*System I: total surfactant conc. = 0.56 wt %, System II: total surfactant conc. = 0.68 wt %.
**Quick separation: separated in few minutes except described otherwise.

### EXAMPLE 3

A ternary surfactant mixture solution, prepared in either deionized water or actual contaminated site groundwater, included sodium dioctyl sulfosuccinate (SEHSS, or AOT), sodium dihexyl sulfosuccinate (MA), and sorbitan monooleate (TWEEN® 80), Sorbitan monostearate (TWEEN® 60), or similar biodegradable nonionic surfactants (see Table 2). We were able to achieve the low IFT microemulsions for the weathered gasoline fuels using a salt additive, NaCl, at concentrations between 1% and 1.4% based on the weight of water in the mixture. Note that the equilibration times for the resulting low IFT microemulsions (as denoted as Type III system) are typically in minutes. Further improvement made was that all of the total surfactant concentrations were between 0.73 wt % to 0.83 wt %. In this example, all three surfactants, SEHSS, MA, and TWEEN® 80 or TWEEN® 60 are readily biodegraded in the environment. Based on this example, a mixture of sodium dioctyl sulfosuccinate (SEHSS), sodium dihexyl sulfosuccinate (MA), and sorbitan monooleate (TWEEN® 80) or Sorbitan monostearate (TWEEN® 60) provided further improvement in equilibration (or separation) time of the low IFT microemulsions as compared to the binary system, dioctyl sulfosuccinate and sodium laureth sulfate SDES-3 (modified) or STEOL® KS-460 or other STEOL® surfactants presented herein.

TABLE 2

| | | Nonionic | | | |
|---|---|---|---|---|---|
| | | sorbitan | | | |
| | MA | sur- | | | |
| SEHSS | (wt | factant | NaCl | Winsor | |
| (wt %) | %) | (wt %) | (wt %) | Type | Note |
| System III* | | | | | |
| 0.2 | 0.4 | 0.1 | 1 | I | Took about 10 min |
| | | | | | to separate |
| 0.23 | 0.4 | 0.1 | 1 | I | Took about 5 min |
| | | | | | to separate |
| 0.23 | 0.4 | 0.1 | 1.4 | III | Quick separation |
| System IV* | | | | | |
| 0.25 | 0.48 | 0.1 | 1 | III | |
| 0.25 | 0.4 | 0.15 | 1 | I | slow separation** |
| 0.3 | 0.48 | 0.15 | 1 | I | slow separation |

All surfactant systems were tested with a weathered gasoline from an actual site, L*.
*System III: POE (20) sorbitan monooleate-Tween 80, System IV: POE (20) sorbitan monostearate-Tween 60.
**Slow separation: separated in more than few hours except described otherwise.

### EXAMPLE 4

A high performance/experimental surfactant, branched alkyl (C₁₄-C₁₅) propyloxylated (8PO) sulfate surfactant (29.6% active, Ave. MW=715) was introduced to the binary SEHSS/MA system to assess the feasibility of creating the low IFT microemulsions (see Table 3). Similarly, addition of the branched alkyl (C₁₄-C₁₅) propyloxylated (8PO) sulfate (Alfoterra® 58) also resulted in low IFT microemulsions in conjunction with SEHSS/MA system. Also, the required amounts of Alfoterra® 58 are similar to other surfactants used in the present invention (=0.1 wt %) and significantly lower than the amounts used by others in prior art.

TABLE 3

| | | branched alkyl | | | |
|---|---|---|---|---|---|
| | | (C₁₄-C₁₅) | | | |
| SEHSS | MA | propyloxylated | NaCl | Winsor | |
| (wt %) | (wt %) | sulfate (wt %) | (wt %) | Type | Note |
| 0.2 | 0.4 | 0.1 | 1 | I | Slow |
| | | | | | separation |
| 0.23 | 0.4 | 0.1 | 0.6 | I | Took about |
| | | | | | 30 min to |
| | | | | | separate |
| 0.23 | 0.4 | 0.1 | 1 | III | Quick |
| | | | | | separation |
| 0.21 | 0.4 | 0.1 | 1 | III | Quick |
| | | | | | separation |
| 0.21 | 0.42 | 0.1 | 1 | III | Quick |
| | | | | | separation |
| 0.21 | 0.44 | 0.1 | 1 | III | Quick |
| | | | | | separation |

### EXAMPLE 5

We further investigated a variety of gasoline fuel samples retrieved from different sites for the selected ternary SEHSS/MA/STEOL® KS-460 system (see Table 4). Also, we added a second salt, CaCl₂, to assess the robustness of this ternary surfactant mixture for creating the low IFT microemulsions. The improved ternary surfactant mixtures (SEHSS/MA/STEOL® KS-460) could produce the desirable low IFT microemulsions for various weathered gasoline samples with both NaCl and CaCl₂ as amendments. One of the advantages of adding CaCl₂ in the surfactant formulations is to minimize the cation exchange in the subsurface soil as a result of high NaCl concentrations injected, and affect the performance of oil recovery. Depending on the site-specific contaminants, some minor adjustments of the surfactant ratios will be necessary to achieve the low IFT microemulsions.

TABLE 4

| | | | SDES-3 | | | | |
|---|---|---|---|---|---|---|---|
| | | MA | (modi- | NaCl | CaCl2 | | |
| | SEHSS | (wt | fied), | (wt | (wt | Winsor | |
| NAPL | (wt %) | %) | (wt %) | %) | %) | Type | Note |
| Site S* | 0.25 | 0.52 | 0.21 | 0.2 | 0.06 | I | 5 min to |
| gasoline | | | | | | | separate |
| | 0.25 | 0.52 | 0.21 | 0.5 | 0.06 | I | |
| | 0.25 | 0.52 | 0.21 | 1 | 0.06 | III | |
| | 0.3 | 0.4 | 0.25 | 1 | 0.05 | I | 5 min to |
| | | | | | | | separate |
| Site C* | 0.25 | 0.52 | 0.21 | 0.4 | 0.18 | | |
| gasoline | 0.25 | 0.52 | 0.21 | 0.5 | 0.18 | I −> III | 5 min to |
| | | | | | | | separate |
| | 0.25 | 0.52 | 0.21 | | | | |
| Site L* | 0.25 | 0.52 | 0.21 | 0.5 | 0.18 | III | |
| gasoline | 0.3 | 0.4 | 0.25 | 0.6 | 0.14 | I | 30 min |
| | | | | | | | to |
| | | | | | | | separate |
| | 0.3 | 0.4 | 0.25 | 0.7 | 0.17 | III | |
| Site | 0.25 | 0.52 | 0.21 | 0.5 | 0.18 | III | |
| SW* | 0.25 | 0.52 | 0.21 | 0.6 | 0.15 | I | 60 min |
| gasoline | | | | | | | to |
| | | | | | | | separate |
| | 0.25 | 0.52 | 0.21 | 0.5 | 0.17 | | |
| | 0.25 | 0.52 | 0.21 | 0.6 | 0.17 | I −> III | 5 min to |
| | | | | | | | separate |
| | 0.3 | 0.4 | 0.25 | 0.6 | 0.13 | I −> III | 13 min |
| | | | | | | | to |
| | | | | | | | separate |
| | 0.3 | 0.4 | 0.25 | 0.7 | 0.15 | III | |
| | 0.3 | 0.4 | 0.25 | 0.7 | 0.17 | III | |

### EXAMPLE 6

In this example, we also studied the feasibility of creating the low IFT microemulsions for different diesel fuels recovered from the contaminated sites using the ternary surfactant mixtures of AOT/MA/TWEEN® 80. TWEEN® 80 is a nonionic sorbitan surfactant available from Croda International Plc. Using the ternary mixture AOT/MA/TWEEN® 80, with some minor adjustments of the surfactant ratios and the amount of salts added, one can quickly produce the desired microemulsions for three diesel fuels tested in the present invention (Table 5). Addition of other sorbitan surfactants with similar properties or molecular structures (e.g., TWEEN® 60, TWEEN® 20) should provide the desired microemulsion as well.

TABLE 5

| | | | Nonionic | | | |
|---|---|---|---|---|---|---|
| | | | sorbitan | | | |
| | | | surfactant, | | | |
| | | | TWEEN ® | | | |
| | SEHSS | MA | 80 | NaCl | CaCl2 | Winsor |
| NAPL | (wt %) | (wt %) | (wt %) | (wt %) | (wt %) | Type |
| E* Site | 0.4 | 0.27 | 0.25 | 1.3 | 0.05 | III |
| Diesel | | | | | | |
| T* Site | 0.4 | 0.27 | 0.25 | 1.3 | 0.05 | III |
| Diesel | | | | | | |
| A* Site | 0.4 | 0.27 | 0.25 | 1.3 | 0.05 | II |
| Diesel | | | | | | |
| | 0.25 | 0.48 | 0.25 | 1 | 0.05 | III |

### EXAMPLE 7

Additional ternary surfactant mixtures, SEHSS/MY-65/STEOL® KS-460 and SEHSS/MA/MY-65, were used to produce the low IFT microemulsions shown in Table 6. AAY-65 is sodium diamyl sulfosuccinate available from the Cytec Industries. Both of these surfactant mixtures create a very low IFT microemulsion for the selected gasoline. Though formation of low IFT microemulsion might be challenging using sodium diamyl sulfosuccinate, AAY-65, in low concentrations as previously described, we were able to incorporate the AAY-65 into our improved ternary surfactant and provide a good alternative formulation to achieve low IFT microemulsion for gasoline fuels and other contaminants.

TABLE 6

| | sodium | | | | | |
|---|---|---|---|---|---|---|
| | diamyl | | | | | |
| | sulfo | | | | | |
| SEHSS | succinate | SDES-3 | NaCl | CaCl2 | Winsor | |
| (wt %) | (wt %) | (wt %) | (wt %) | (wt %) | Type | Note |
| 0.3 | 0.4 | 0.25 | 0.6 | 0.05 | I | <30 min to |
| | | | | | | separate |
| 0.3 | 0.4 | 0.25 | 1 | 0.05 | I −> III | 5 min to |
| | | | | | | separate |
| 0.3 | 0.4 | 0.25 | 1.4 | 0.05 | III | good middle |
| | | | | | | phase |

| | | sodium | | | |
|---|---|---|---|---|---|
| | | diamyl | | | |
| | | sulfo | | | |
| SEHSS | MA | succinate | NaCl | CaCl2 | Winsor |
| (wt %) | (wt %) | (wt %) | (wt %) | (wt %) | Type |
| 0.3 | 0.4 | 0.1~0.3 | 1 | 0 | II |
| 0.3 | 0.4 | 0.35 | 0.5 | 0 | III |

S* site weathered gasoline was used in this study.

### EXAMPLE 8

In addition to laboratory batch experiments, one-dimensional soil column tests were conducted to evaluate the oil (gasoline) recovery under hydrodynamic conditions for surfactant mixtures SEHSS/MA/TWEEN® 80 and SEHSS/MA/STEOL® KS-46 as presented in this example. Results of these soil column tests indicated that both low concentration ternary surfactant mixtures, SEHSS/MA/TWEEN® 80 and SEHSS/MA/STEOL® KS-46, could remove most of the oil from the soil-packed column (see Table 7). SEHSS/MA/STEOL® KS-46 provides better oil recovery compared to SEHSS/MA/TWEEN® 80. The lesser oil volume recovered (between 80 to 86%) by the SEHSS/MA/TWEEN® 80 system might be due to somewhat higher sorption losses of the anionic/anionic/nonionic system.

TABLE 7

| | | | Nonionic | | | | |
|---|---|---|---|---|---|---|---|
| | | | sorbitan | | | | |
| | | | sur- | | | | |
| | | | factant, | | | | |
| | | MA | TWEEN ® | NaCl | | | Oil |
| | SEHSS | (wt | 80 | (wt | CaCl2 | | Removal |
| NAPL | (wt %) | %) | (wt %) | %) | (wt %) | PV | (%) |
| Sw* | 0.25 | 0.48 | 0.25 | 1.4 | 0.06 | 2 | 85 |
| gasoline | | | | | | | |
| L* | 0.25 | 0.48 | 0.25 | 1.4 | 0.06 | 2 | 86 |
| gasoline | | | | | | | |
| C* | 0.25 | 0.45 | 0.25 | 1.4 | 0.07 | 2 | 86 |
| gasoline | | | | | | | |
| S* | 0.25 | 0.7 | 0.25 | 1 | 0.05 | 2 | 80 |
| gasoline | | | | | | | |

| | | | | | | | Oil |
|---|---|---|---|---|---|---|---|
| | SEHSS | MA | SDES-3 | NaCl | CaCl2 | | Removal |
| NAPL | (wt %) | (wt %) | (wt %) | (wt %) | (wt %) | PV | (%) |
| C* | 0.25 | 0.48 | 0.11 | 1 | 0.05 | 2 | 89 |
| gasoline | | | | | | | |
| S* | 0.25 | 0.52 | 0.21 | 1 | 0.05 | 2 | 91 |
| gasoline | | | | | | | |

Soils used in these columns were collected from individual sites.

From the above description, it is clear that the present invention is well adapted to carry out the objects and to attain the advantages mentioned herein as well as those inherent in the invention. While presently preferred embodiments of the invention have been described for purposes of this disclosure, it will be understood that numerous changes may be made which will readily suggest themselves to those skilled in the art and which are accomplished within the spirit of the invention disclosed.

What is claimed is:

1. A surfactant system comprising: sodium bis(2-ethylhexyl) sulfosuccinate surfactant, a laureth sulfate anionic surfactant, and water, wherein the surfactants are present in the system in concentrations effective to produce a microemulsion, without the need for a cosolvent or linking molecule, when the surfactant system is combined with an oil.

2. The surfactant system of claim 1 wherein the surfactant system has a total surfactant concentration of less than about 1.0 wt % based on weight of water present in the surfactant system.

3. The surfactant system of claim 1 wherein the laureth sulfate anionic surfactant is selected from the group consisting of sodium dodecyl ethoxy (1) sulfate, sodium dodecyl ethoxy (2) sulfate, sodium dodecyl ethoxy (3) sulfate, sodium dodecyl ethoxy (4) sulfate, and mixtures thereof.

4. The surfactant system of claim 1, further comprising sodium dihexyl sulfosuccinate surfactant.

5. The surfactant system of claim 4 wherein the surfactant system has a total surfactant concentration of less than about 1.5 wt % based on weight of water present in the surfactant system.

6. The surfactant system of claim 4 wherein the surfactant system has a total surfactant concentration of less than about 1.0 wt % based on weight of water present in the surfactant system.

7. The surfactant system of claim 4 wherein the oil of the microemulsion is selected from the group consisting of crude oil, petroleum products, organic solvents, organic contaminants, pharmaceutical compositions, and edible oils (e.g., vegetable oil), fats and greases.

8. The surfactant system of claim 4 wherein the microemulsion is a Windsor Type III middle phase microemulsion.

9. The surfactant system of claim 8 wherein the Windsor Type III middle phase microemulsion has a separation time less than about 2 hours.

10. The surfactant system of claim 8 wherein the Windsor Type III middle phase microemulsion has a separation time less than about 15 minutes.

11. The surfactant system of claim 1, further comprising sodium diamyl sulfosuccinate surfactant.

12. The surfactant system of claim 11 wherein the surfactant system has a total surfactant concentration of less than about 1.5 wt % based on the weight of water present in the surfactant system.

13. The surfactant system of claim 11 wherein the surfactant system has a total surfactant concentration of less than about 1.0 wt % based on the weight of water present in the surfactant system.

14. The surfactant system of claim 11 wherein the oil of the microemulsion is selected from the group consisting of crude oil, petroleum products, organic solvents, organic contaminants, pharmaceutical compositions, and edible oils (e.g., vegetable oil), fats and greases.

15. The surfactant system of claim 11 wherein the emulsion is a Windsor Type III middle phase microemulsion.

16. The surfactant system of claim 15 wherein the Windsor Type III middle phase microemulsion has a separation time less than about 2 hours.

17. The surfactant system of claim 15 wherein the Windsor Type III middle phase microemulsion has a separation time less than about 15 minutes.

18. The surfactant system of claim 1 wherein the water is selected from the group consisting of fresh water and salt water.

19. The surfactant system of claim 18 wherein the salt water is selected from the group consisting of electrolyte, brine, seawater, formation water, and combinations thereof.

20. The surfactant system of claim 1 wherein the water includes NaCl and CaCl₂, wherein the NaCl is present in an amount of up to about 5 wt % based on the total weight of water, and the CaCl₂ is present in an amount of up to about 1 wt % based on the total weight of water.

21. The surfactant system of claim 1 wherein the microemulsion is a Windsor Type III middle phase microemulsion.

22. A surfactant system comprising: water, sodium bis(2-ethylhexyl)sulfosuccinate surfactant, sodium dihexyl sulfossucinate surfactant, and a third surfactant selected from the group consisting of a branched alkyl (C₁₄-C₁₅) propyloxylated sulfate surfactant, sodium diamyl sulfosuccinate surfactant, polyoxyethylene (20) sorbitan monooleate surfactant, polyoxyethylene (20) sorbitan monostearate surfactant, and mixtures thereof, wherein the surfactants are present in the system in concentrations effective to produce a microemulsion, without the need for a cosolvent or linking molecule, when the surfactant system is combined with an oil.

23. The surfactant system of claim 22 wherein the surfactant system has a total surfactant concentration of less than about 1.5 wt % based on weight of water present in the surfactant system.

24. The surfactant system of claim 22 wherein the third surfactant is a branched alkyl (C₁₄-C₁₅) propyloxylated sulfate surfactant and the surfactant system has a total surfactant concentration of less than about 1.0 wt % based on weight of water present in the surfactant system.

25. The surfactant system of claim 24 wherein the oil of the microemulsion is selected from the group consisting of crude oil, petroleum products, organic solvents, organic contaminants, pharmaceutical compositions, and edible oils (e.g., vegetable oil), fats and greases.

26. The surfactant system of claim 24 wherein the microemulsion is a Windsor Type III middle phase microemulsion and wherein the Windsor Type III middle phase microemulsion has a separation time of less than about 2 hours.

27. The surfactant system of claim 22 wherein the third surfactant is sodium diamyl sulfosuccinate and the surfactant system has a total surfactant concentration of less than about 1.0 wt % based on weight of water present in the surfactant system.

28. The surfactant system of claim 27 wherein the oil of the microemulsion is selected from the group consisting of crude oil, petroleum products, organic solvents, organic contaminants, pharmaceutical compositions, and edible oils (e.g., vegetable oil), and fats and greases.

29. The surfactant system of claim 27 wherein the microemulsion is a Windsor Type III middle phase microemulsion having a separation time of less than about 2 hours.

30. The surfactant system of claim 22 wherein the third surfactant is polyoxyethylene (20) sorbitan monooleate and the surfactant system has a total surfactant concentration of less than about 1.0 wt % based on weight of water present in the surfactant system.

31. The surfactant system of claim 30 wherein the oil is selected from the group consisting of crude oil, petroleum products, organic solvents, organic contaminants, pharmaceutical compositions, and edible oils (e.g., vegetable oil), fats and greases.

32. The surfactant system of claim 30 wherein the microemulsion is a Windsor Type III middle phase microemulsion having a separation time of less than about 2 hours.

33. The surfactant system of claim 22 wherein the third surfactant is polyoxyethylene (20) sorbitan monostearate and the surfactant system has a total surfactant concentration of less than about 1.0 wt % based on weight of water present in the surfactant system.

34. The surfactant system of claim 33 wherein the oil is selected from the group consisting of crude oil, petroleum products, organic solvents, organic contaminants, pharmaceutical compositions, and edible oils (e.g., vegetable oil), fats and greases.

35. The surfactant system of claim 33 wherein the microemulsion is a Windsor Type III middle phase microemulsion having a separation time of less than about 2 hours.

36. The surfactant system of claim 22 wherein the water is selected from the group consisting of fresh water and salt water.

37. The surfactant system of claim 36 wherein the salt water is selected from the group consisting of electrolyte, brine, seawater, formation water, and combinations thereof.

38. The surfactant system of claim 22 wherein the water includes NaCl and CaCl₂, wherein the NaCl is present in an amount of up to about 5 wt % based on the total weight of water, and the CaCl₂ is present in an amount of up to about 1 wt % based on the total weight of water.

39. The surfactant system of claim 22 wherein the microemulsion is a Windsor Type III middle phase microemulsion.

40. A method for substantially removing contaminants from a subsurface formation, the method comprising the steps of:
introducing into the subsurface formation a surfactant system comprising water and a surfactant mixture, the surfactant mixture selected from the group consisting of (a) sodium bis(2-ethylhexyl)sulfosuccinate and sodium laureth sulfate; (b) sodium bis(2-ethylhexyl)sulfosuccinate, sodium laureth sulfate and sodium dihexyl sulfosuccinate; (c) sodium bis(2-ethylhexyl) sulfosuccinate, sodium laureth sulfate and sodium diamyl sulfosuccinate; and (d) sodium bis(2-ethylhexyl)sulfosuccinate, sodium dihexyl sulfossucinate, and a third surfactant, the third surfactant selected from the group consisting of a branched alkyl (C₁₄-C₁₅) propyloxylated sulfate surfactant, sodium diamyl sulfosuccinate, polyoxyethylene (20) sorbitan monooleate, polyoxyethylene (20) sorbitan monostearate, and mixtures thereof;
wherein the surfactants are present in the surfactant system in concentrations effective to produce a microemulsion, without the need for a cosolvent or linking molecule, when the surfactant system is combined with the contaminants;
contacting the surfactant system with the contaminants to form a microemulsion; and
recovering the resulting microemulsion.

41. A process for recovering crude oil from a subterranean oil-bearing formation comprising, comprising the steps of:
injecting a surfactant system into the subterranean oil-bearing formation, the surfactant system comprising water and a surfactant mixture, the surfactant mixture selected from the group consisting of (a) sodium bis(2-ethylhexyl)sulfosuccinate and sodium laureth sulfate; (b) sodium bis(2-ethylhexyl) sulfosuccinate, sodium laureth sulfate and sodium dihexyl sulfosuccinate; (c) sodium bis(2-ethylhexyl)sulfosuccinate, sodium laureth sulfate and sodium diamyl sulfosuccinate; and (d) sodium bis(2-ethylhexyl)sulfosuccinate, sodium dihexyl sulfossucinate, and a third surfactant, the third surfactant selected from the group consisting of a branched alkyl (C₁₄-C₁₅) propyloxylated sulfate surfactant, sodium diamyl sulfosuccinate, polyoxyethylene (20) sorbitan monooleate, polyoxyethylene (20) sorbitan monostearate, and mixtures thereof;
wherein the surfactants are present in the surfactant system in concentrations effective to produce a microemulsion, without the need for a cosolvent or linking molecule, when the surfactant system is combined with the crude oil;
injecting a drive fluid into the formation and driving the crude oil toward at least one recovery well; and
recovering the crude oil via the at least one recovery well.

42. A process for cleaning organic contaminants from a hard surface comprising:
contacting the hard surface with an effective amount of a surfactant system comprising water and a surfactant mixture, the surfactant mixture selected from the group consisting of (a) sodium bis(2-ethylhexyl)sulfosuccinate and sodium laureth sulfate; (b) sodium bis(2-ethylhexyl)sulfosuccinate, sodium laureth sulfate and sodium dihexyl sulfosuccinate; (c) sodium bis (2-ethylhexyl)sulfosuccinate, sodium laureth sulfate and sodium diamyl sulfosuccinate; and (d) sodium bis(2-ethylhexyl)sulfosuccinate, sodium dihexyl sulfossucinate, and a third surfactant, the third surfactant selected from the group consisting of a branched alkyl (C₁₄-C₁₅) propyloxylated sulfate surfactant, sodium diamyl sulfosuccinate, polyoxyethylene (20) sorbitan monooleate, polyoxyethylene (20) sorbitan monostearate, and mixtures thereof,
wherein the surfactants are present in the surfactant system in concentrations effective to produce a microemulsion, without the need for a cosolvent or linking molecule, when the surfactant system is combined with the organic contaminants.

43. A method for laundering fabrics having organic contaminants, which method includes the step of diluting, in an aqueous bath, an aqueous liquid composition in its neat form, said liquid composition comprising a surfactant mixture selected from the group consisting of (a) sodium bis(2-ethylhexyl)sulfosuccinate and sodium laureth sulfate; (b) sodium bis(2-ethylhexyl)sulfosuccinate, sodium laureth sulfate and sodium dihexyl sulfosuccinate; (c) sodium bis(2-ethylhexyl)sulfosuccinate, sodium laureth sulfate and sodium diamyl sulfosuccinate; and (d) sodium bis(2-ethylhexyl)sulfosuccinate, sodium dihexyl sulfossucinate, and a third surfactant, the third surfactant selected from the group consisting of a branched alkyl (C₁₄-C₁₅) propyloxylated sulfate surfactant, sodium diamyl sulfosuccinate, polyoxyethylene (20) sorbitan monooleate, polyoxyethylene (20) sorbitan monostearate, and mixtures thereof,
wherein the surfactants become present in the aqueous bath in concentrations effective to produce a microemulsion, without the need for a cosolvent or linking molecule, when the surfactant system is combined with the organic contaminants.

44. A method for intravenous drug delivery, comprising: preparing microdroplets of the drug with a surfactant system, the surfactant system comprising water and a surfactant mixture, the surfactant mixture selected from the group consisting of (a) sodium bis(2-ethylhexyl)sulfosuccinate and sodium laureth sulfate; (b) sodium bis(2-ethylhexyl)sulfosuccinate, sodium laureth sulfate and sodium dihexyl sulfosuccinate; (c) sodium bis(2-ethylhexyl)sulfosuccinate, sodium laureth sulfate and sodium diamyl sulfosuccinate; and (d) sodium bis(2-ethylhexyl)sulfosuccinate, sodium dihexyl sulfossucinate, and a third surfactant, the third surfactant selected from the group consisting of a branched alkyl (C₁₄-C₁₅) propyloxylated sulfate surfactant, sodium diamyl sulfosuccinate, polyoxyethylene (20) sorbitan monooleate, polyoxyethylene (20) sorbitan monostearate, and mixtures thereof,
wherein the surfactants are present in the surfactant system in concentrations effective to produce a microemulsion, without the need for a cosolvent or linking molecule, when the surfactant system is combined with the drug.
